(19) United States
(12) Patent Application Publication
(10) Pub. No.: US 2009/0113982 A1
(43) Pub. Date: May 7, 2009

(54) Multi-dimensional explosive detector

(76) Inventors: Robert Hodyss, Pasadena, CA (US); Jesse L. Beauchamp, LaCanada Flintridge, CA (US)

Correspondence Address: CHRISTIE, PARKER & HALE, LLP, PO BOX 7068, PASADENA, CA, 91109-7068, US

(21) Appl. No.: 11/364,718

(22) Filed: Feb. 27, 2006

Related U.S. Application Data
(60) Provisional application No. 60/656,211, filed on Feb. 25, 2005.

(51) Int. Cl. G01N 33/22; G01N 30/02; G01J 3/28; G01J 5/02; H01J 49/26; G01N 1/22; G01N 25/18; G01N 27/62; G01N 29/02

(52) U.S. Cl. 73/1.06; 73/23.41; 356/326; 250/339.08; 250/281; 73/25.03; 324/468; 73/590; 73/863.12

(57) ABSTRACT

A system and methodology for the trace detection of organic explosives is described. The detector system combines a separation system, such as a gas chromatograph to separate the components of an explosive mixture, with a pyrolysis detector. In operation, effluent from the separation system is pyrolyzed and the fragments produced on pyrolysis of the explosive compound are then detected. The small molecule fragments exhibit sharply banded, characteristic spectrum, enabling detection of the explosive materials. The system is tested using the explosive materials nitrobenzene and 2,4-dinitrotoluene, and with the nitramine explosive tetryl. Detection limits are 25 ng for nitrobenzene, and 50 ng for 2,4-dinitrotoluene. Tetryl is detected with a detection limit of 50 ng.

### CROSS-REFERENCE TO RELATED APPLICATIONS

This application claims priority based on U.S. provisional application No. 60/656,211, filed Feb. 25, 2005, the disclosure of which is incorporated herein by reference.

### STATEMENT OF FEDERALLY FUNDED RESEARCH

This invention was made with Government support under a grant from the National Aeronautics and Space Administration administered by the Jet Propulsion Laboratory. The Government has certain rights in this invention.

### FIELD OF THE INVENTION

The current invention is directed to a system and method for the detection of explosives; and more particularly to a multi-dimensional detection system and method based on the ultraviolet detection of molecules produced in the thermal decomposition of explosive compounds separated by gas chromatography.

### BACKGROUND OF THE INVENTION

Systems and methods for detecting explosives are urgently needed, and are now at the forefront of many research efforts. An ideal explosives detection system would be reliable, simple and provide an unambiguous signal when explosives are detected. However, detection of explosives is complicated for a variety of fundamental physical and chemical reasons. First, the vapor pressure of most common explosives is vanishingly small. See, e.g., B. C. Dionne, et al., J. Energetic Mat. 4, 447 (1986), the disclosure of which is incorporated herein by reference. As a result, methods that rely on sampling of air spaces need to either sample very large volumes or have exceedingly small detection limits. Second, composite explosive materials actually serve to suppress the already small vapor pressures of these explosive materials. Third, explosive materials are easily packaged in air-tight containers that can effectively reduce the vapor pressure by a factor of 1000, for by example sealing the materials in plastics. Finally, interferences from solvents or plastics can lead to false alarms that are difficult to distinguish from actual positive tests.

Current explosives detection methodologies attempt to overcome many of these problems by making use of the fragmentation of the target molecules, followed by sensitive detection of the released gaseous products. Since many explosive compounds are based on nitroorganics, NO is a common product of decomposition, and a good target for sensitive detection. For example, groups have recently reviewed the wide variety of techniques used to detect explosives, and NO has been detected as the product of thermal decomposition of nitroorganics by IR spectroscopy, microwave spectroscopy, and fluorescence. In addition, a number of non-optical techniques, such as mass spectrometry, have also been used. (See, e.g., J. I. Steinfeld, et al., Annu. Rev. Phys. Chem. 49, 203-232 (1998); & D. S. Moore, S. Rev. Sci. Instrum. 75, 2499-2512 (2004); the disclosures of which are incorporated herein by reference.)

One of the more successful techniques to detect NO is the use of chemiluminescence. The EGIS system, manufactured by Thermo Electron Corporation, utilizes this type of detector. (This system is discussed in D. H. Fine, et al., SPIE Subst. Detect. Syst. 2092, 131-136 (1993); & D. H. Fine, et. al., Anal. Chem. 47, 1188-1191 (1975), the disclosures of which are incorporated herein by reference.) The chemiluminescence detector, also known as a thermal energy analyzer, operates by pyrolyzing the sample in a catalytic reactor to release NO. The NO is subsequently reacted with ozone to produce excited NO which emits infrared radiation that is detected with a photomultiplier. The EG1S system is selective for nitroorganics and is highly sensitive, able to respond to a few picograms of analyte. However, this system is highly complex, requiring among other things a generator or storage source for ozone, which itself is highly toxic and explosive.

Another method that has been proposed is a multidimensional test which couples gas phase ultraviolet absorption with gas chromatography. Some form of this system has been practiced sporadically for the past 40 years. (See, e.g., W. Kaye, Anal. Chem. 34, 287-293 (1962); T. Cedron-Fernandez, et al., Talanta 57, 555-563 (2000); H. V. Lagesson, et al., Chromatographia 52, 621-630 (2000); V. Lagesson, et al., J. Chromatogr. 867, 187-206 (2000); M. J. McQuaid, et al., Appl. Spectrosc. 45, 916-917 (1991); A. D. Usachev, et al., Appl. Spectrosc. 55, 125-129 (2001); and W. A. Schroeder, et al., Anal. Chem. 23, 1740-1747 (1951), the disclosures of which are all incorporated herein by reference.) Kaye reported the first GC-UV system in 1962, which used ultraviolet absorption at 170 nm for the analysis of a chromatographic separation of gasoline. GC-UV systems have since been used for the analysis of wine, indoor dust, and proposed as a means for functional group analysis. The nitroorganic explosives possess strong absorptions in the V, and their direct detection by GC-UV is possible. However, the spectra are broad and featureless, and overlap with the absorptions of many other organic compounds. As a result, the ultraviolet absorption spectra of the nitroorganic explosives themselves cannot provide unambiguous detection of explosives in the presence of other organics.

In addition, most of these systems provide the capability to detect only nitrogen containing explosives. Such techniques would be unable to detect explosives such as triacetone triperoxide, which, due to its ease of manufacture, has been used in a number of terrorist attacks. Accordingly, an improved system that allows for the fast, accurate and simple detection of a wide variety of explosive materials is needed.

### SUMMARY OF THE INVENTION

The current invention is directed to a system and method for the multidimensional detection of explosives.

In one embodiment, the explosives detector includes a separator having a fluid passage with an inlet and an outlet, said inlet being in fluid communication with a sample having at least two distinct components, said separator being designed to pass each component of the sample through the fluid passage at a rate dependent on the physical properties of the component such that each of the components from the sample pass through the outlet of the separator at a different time.

In another embodiment, the explosives detector includes a pyrolysis detector including a pyrolyzer having a heated element capable of decomposing each component into a plurality of molecular fragments, and a detector in fluid communication with the pyrolyzer such that each molecular fragment is identified by the detector.

In still another embodiment, the explosives detector includes an analyzer in signal communication with at least the separator and the pyrolysis detector such that the time data from the separator and the fragment data from the pyrolysis detector are analyzed to provide a multidimensional data set indicative of the presence of an explosive material in the sample.

In yet another embodiment, the separator is a gas chromatograph.

In yet another embodiment, the pyrolyzer is a Nichrome wire, or a catalytic pyrolyzer.

In still yet another embodiment, the pyrolysis detector is a spectroscopic detector such as an ultraviolet detector.

In still yet another embodiment, the explosives detector includes a secondary detector in fluid communication with the outlet of the separator, such that each separated component of the sample is identified prior to pyrolysis. In such an embodiment, the secondary detector is may be selected from the group consisting of infrared (IR), Fourier transform infrared (FTIR), mass spectroscopy (MS), electron capture (ECD), chemiluminescence or thermal energy analysis (TEA), flame ionization (FI), thermal conductivity (TC), and surface acoustic wave (SAW). Also in such an embodiment, the secondary detector is in signal communication with the analyzer to provide component data on each separated component of the sample to the multidimensional data set.

In still yet another embodiment, the explosives detector includes a sample preconcentrator. In such an embodiment, the preconcentrator may include an enclosed volume having a sample absorbent material in contact with a flash heating system such that the sample is first absorbed onto the absorbent material and then flash heated within the enclosed volume to create a concentrated volume of sample, or a particle collector.

In still yet another embodiment, the analyzer includes a stored calibration standard for one of either the qualitative or quantitative analysis of the sample.

In still yet another embodiment, the analyzer includes at least on signal processing algorithm for processing the multidimensional data set.

In another embodiment, the invention is directed to a method for detecting explosives using a multidimensional detector.

### BRIEF DESCRIPTION OF THE DRAWINGS

These and other features and advantages of the present invention will be better understood by reference to the following detailed description when considered in conjunction with the accompanying drawings wherein:

FIG. 1 provides a schematic diagram of the basic detection steps or systems in accordance with an exemplary embodiment of the current invention;

FIG. 2 provides a block diagram of an exemplary multidimensional detector system, and particularly a gas chromatography-pyrolysis-ultraviolet detector (GC-PUD) system, the inset shows details of the pyrolyzer;

FIG. 3 shows data taken from an exemplary multidimensional detector system, including a) a chromatogram of 500 ng each of nitrobenzene (elutes at 395 s) and 2, 4-dinitrotoluene (620 s), b) an ultraviolet spectrum obtained at 100 s, showing ammonia formed on the pyrolysis of acetonitrile, c) an ultraviolet spectrum obtained at 395 s, showing NO formed on the pyrolysis of nitrobenzene, and d) an ultraviolet spectrum obtained at 620 s, showing NO formed on the pyrolysis of 2, 4-dinitrotoluene; and

FIG. 4 provides a log scale plot of peak area vs. mass of analyte injected for nitrobenzene (circles), 2,4-dinitrotoluene (squares) and tetryl (triangles).

### DETAILED DESCRIPTION OF THE INVENTION

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The unique properties of high explosives originate from the presence, in the same molecule, of fuel and oxidizer. This proximity leads to the ability to achieve extremely high reaction rates because diffusion is not the rate-limiting step. Although nitrogen is present in nearly all high explosive compounds, this is not always the case as strained ring or other structures with high enthalpy of formation can also react explosively. Table 1, below provide an incomplete list of some of the common explosives that are the subject of this invention.

TABLE 1
List of Common Explosives

| Abbreviation | Common Name | Chemical Structure |
|---|---|---|
| Amatol | Amatol | mixture of AN and TNT |
| AN | ammonium nitrate | NH₄NO₃ |
| AP | ammonium perchlorate | NH₄ClO₄ |
| ANFO | ANFO | composition of AN and fuel oil |
| A-3 | Comp A-3 | composition of RDX and heavy wax |
| Comp B | Comp B | composition of 60% RDX and 40% TNT, |
| | | optionally with wax |
| C-4 | Comp C-4 | composition of 91% RDX plus waxes and oils |
| Cyclotol | Cyclotol | composition of 75% RDX and 25% TNT |
| DDNP | Dinol | diazodinitrophenol |
| DEGDN | DEGDN | diethyleneglycol dinitrate |
| Detasheet | Detasheet | composition of PETN and NC with plasticizers |
| DNB | DNB | 1,3-dinitrobenzene |
| EGDN | nitroglycol | ethylene glycol dinitrate |
| H-6 | H-6 | composition of RDV and TNT with aluminum |
| | | particles and wax |
| HBX-1 | HBX-1 | composition of RDX and TNT with aluminum |
| | | particles and wax |
| HMTD | HMTD | hexamethylenetriperoxidediamine |
| HMX | octagen | octahydro-1,3,5,7-tetronitro-1,3,5,7-tetrazocine |
| HNS | hexanitrostilbene | 1,1′-(1,2-ethenediyl)bis-[2,4,6-trinitrobenzene] |
| HNAB | HNAB | hexanitro-azobenzene |
| LX-10 | LX-10 | PBX with HMX and binding agent |
| LX-17 | LX-17 | PBX with TATB and bonding agent |
| MATB | ammonium picrate | monoamine-trinitro-benzene |
| NB | NB | nitrobenzene |
| NC | gun cotton | nitro cellulose |
| NG | RNG, nitroglycerine, | glyceryl trinitrate |
| | nitro | |
| Octol | Octol | composition of 75% HMX and 25% TNT |
| PBX | plastic bonded explosive | N/A |
| PBX-9404 | PBX | PBX with HMX and energetic bonding agents |
| PBX-9501 | PBX | PBX with HMX, bonding agents and plasticizers |
| PE 4 | Britich Comp C | RDX with waxes and/or heavy oils |
| PETN | pentaerythritol | 2.2-bis[(nitroxy)methyl]-1,3-propanediol dinitrate |
| | tetranitrate | |
| Picric acid | Picric acid | 2,4,6-trinitrophenol |
| Pentolite | Pentolite | composition of 50% PETN and 50% TNT |
| RDX | cyclonite, hexogen | hexahydro-1,3,5-trinitro-1,3,5 triazine |
| Semtex-H | Semtex | composition of RDX and PETN with heavy oils |
| | | and rubbers |
| TATB | trinitro-triamino- | 2,4,6-trinitro-1,3,5-benzene-triamine |
| | benzene | |
| TATP | TATP | triacetone triperoxide |
| Tetryl | Tetryl | methyl-2,4,6-trinitophenylnitramine |
| TEGDN | TEGDN | triethyleneglycol dinitrate |
| TNB | TNB | 1,3,5-trinitrobenzene |
| TNT | 2,4,6-trinitrotoluene | 2-methyl-1,3,5-trinitro-benzene |
| Tritonal | Tritonal | aluminized TNT |

As can be surmised from the molecular formulas in the table, most common explosives are rich in nitrogen and oxygen and relatively poor in carbon and hydrogen, with some notable exceptions such as TATP. This fact is often exploited for bulk explosive detectors that look at the oxygen/nitrogen ratio and the anomalously large nitrogen content. However, such techniques are inadequate when attempting to detect tract quantities of explosives. As such, these trace detection methods rely instead on a molecular signature of some kind, such as retention time in chromatography, or unique mass or vibrational spectrum. As discussed previously the problem with these techniques generally is that the spectra for these large complex molecules are often featureless making it difficult, if not impossible to distinguish between explosives and other nitrogen containing compounds.

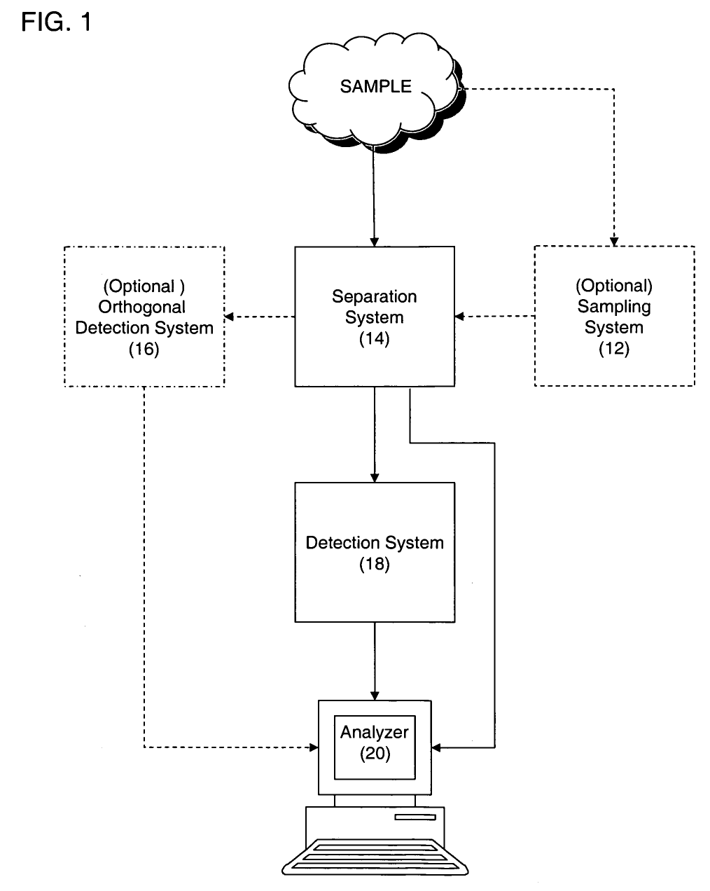

The current invention addresses these deficiencies through a multidimensional approach. Specifically, the current invention is directed to a novel multidimensional system and method for the trace detection of organic explosives that combines a separator such as a conventional gas chromatography and a separate pyrolyzing detector. The general scheme of the current method is shown schematically in FIG. 1. The detection scheme may include an optional sampling methodology or system (12, which may or may not preconcentrate the analyte), a separation step or device (14, which may occur during sampling and/or preconcentration), an optional orthogonal detection method or system (16, referred to herein after as hyphenation), a pyrolysis detection step or system (18); and an analyzer (20) connected to at least the separation device and the pyrolysis detection system. Each of these individual steps and systems will be discussed in greater detail below.

Although conventional techniques have taken a multidimensional approach to explosive detection, even including separation by gas chromatography followed by detection by ultraviolet techniques, these prior art techniques have not been focused on addressing the unique problems in the detection of explosive materials, namely the very low vapor pressures and the broad featureless UV absorption bands of most explosives that overlap many common contaminants. The vapor pressures for most common explosives can be found at Federal Register, 67, No. 81 The current invention addresses the flaws in these past methodologies by first separating the components of a sample, and then controllably pyrolyzing the separated components to form small molecules from the larger organic species. The small molecules formed by the pyrolyzed explosives have sharp distinct UV absorption bands that can then be easily characterized. By combining the results of the retention time measurements and the corresponding spectroscopic measurements a multidimensional fingerprint of each of the molecules in a sample can be obtained. It has been determined that these results provide a highly sensitive, inexpensive method of both the qualitative and quantitative detection of explosive materials. In short, the combination of the separation and detection of fragments of explosive molecules through pyrolysis provides a multidimensional analysis of the sample, and the capability to provide an accurate detection fingerprint for a vast array of explosives.

(Optional) Sampling System

Turning to the sampling system, although the sample system could merely be an inlet into the separator, and the sample could be drawn unprocessed straight from the background environment, because of the very low vapor pressures for most explosive materials, in one embodiment the system also includes a system to preconcentrate the sample to improve the volume detection limit. Such a sampling system can be of any design suitable for collecting and preconcentrating samples for analysis.

In one exemplary embodiment the sampling system may include flowing the sample through a volume having a material disposed within that adheres explosive materials, followed by flash heating the material to desorb the explosive within the volume and then injecting that concentrated material into the detection system. In such an embodiment any material suitable for adhering explosive materials may be used, such as, for example, Teflon, glass, quartz, nickel, stainless steel, gold, platinum, copper, fused silica, aluminum, plastic, etc. In addition, the material may be provided in any form suitable, such as, for example, a fine mesh, membrane, ribbon, long tube, etc. Exemplary system are provided in G. J. Wendel, et al., Proc. Symp. Explosives Detection Technology 2nd, edited by W. H. Makky, Atlantic City, FAA 181-186 (1996); G. S. Settles, et al., Proc. Symp. Explosives Detection Technology 2nd, edited by W. H. Makky, Atlantic City, FAA 65-70 (1996); and J. E. Parmeter, et al., Proc. Symp. Explosives Detection Technology 2nd, edited by W. H. Makky, Atlantic City, FAA 187-192 (1996), the disclosure of which are incorporated herein by reference. Other more exotic high surface area materials may also be used, such as for example, solid phase extraction materials, polymeric materials, and fullerenes. Exemplary suitable materials are discussed, for example, in E. Psillakis, et al. J. Chromatogr., A 907, 211 (2001); E. J. Houser, et al., Talanta 54, 469 (2001); D. C. Stahl, et al., Environ. Sci. Technol. 35, 3507 (2001); and K. G. Furton, et al., J. Chromatogr., A. 885, 419 (2000), the disclosures of which are all incorporated herein by reference.

Such trapping materials may optionally be coated with species-selective coatings to improve the selectivity of the sampler/preconcentrator. For example, in one embodiment, polymers may be used that selectively bind to a nitro functional group of a polynitroaromatic increasing the polymer-nitroaromatic air partition coefficients and hence sensor signals. Some systems have been shown to increase the sensitivity of the detection to <100 parts per trillion by volume (pptv) for DNT. Some exemplary systems are described in the references to Psillakis, Houser, Stahl, and Furton, cited above.

Another exemplary sampling method utilizes the inherent preconcentration found with collecting particles of high explosives. These particles can adhere to surfaces or can be airborne, and a single particle having a diameter as small as 5 μm can contains many molecules of an explosive material as 1 L of equilibrium vapor pressure STP air. In such a system the particles can be collected by vacuuming or otherwise sweeping a volume, of such particles into the system by swiping surfaces of potentially contaminated objects and then placing the swiped samples into an enclosure for analysis. Such sampling methods may also be incorporated into walk-through portals, such as metal detectors that enable the collection of vapors and/or particles from subjects. In such an embodiment, the system may include puffers or air jets, paddles, acoustic energy, and other types of air-flow devices. The collected material may then be input into detection system. Exemplary systems are described by W. McGann, et al., Proc. Int. Symp. Explosive Detection Technology, 1st, Atlantic City, FAA 518-531 (1992); S. F. Hallowell, Talanta 54, 447 (2001); D. C. Seward, et al., First International Symposium on Explosive Detection Technology, Atlantic City, N.J. 441-453 (1991); D. C. Seward, et al., Second Explosives Detection Technology Symposium & Aviation Security Technology Conference, Atlantic City, N.J. 162-169 (1996); C. Rhykerd, et al., Nucl. Mater. Managem. 26, 97 (1997); G. J. Wendel, et al., Second Explosives Detection Technology Symposium & Aviation Security Technology Conference, Atlantic City, N.J. 181-186 (1996); J. E. Parmeter, et al., Second Explosives Detection Technology Symposium & Aviation Security Technology Conference, Atlantic City, N.J. 187-192 (1996); J. R. Hobbs, et al., Advances in Analysis and Detection of Explosives, edited by J. Yinon, Kluwer Academic, Dordrecht, 437-453 (1993); E. E. A. Bromberg, et al., Advances in Analysis and Detection of Explosives, edited by J. Yinon, Kluwer Academic, Dordrecht, 473-484 (1993); M. M. Hintze, et al., First International Symposium on Explosive Detection Technology, Atlantic City, N.J. 634-636 (1991); and A. Jenkins, et al. First International Symposium on Explosive Detection Technology, Atlantic City, N.J. 532-551 (1991), the disclosures of which are incorporated herein by reference.

Separation System

As shown in the schematic provided in FIG. 1, once the sample has been collected it is injected into the separation system. The separation system and step is designed to separate out the components of a sample and to provide retention time information prior to the determination of their identity by spectroscopic means. This allows for the removal of interferences by separating the molecules contained in a sample by mobility. Although any suitable method capable of separating components of a mixed sample may be used, some exemplary systems include gas chromatography, high performance liquid chromatography and capillary electrophoresis.

In the chromatographic methods, a sample is pushed through a column having a stationary phase by a carrier, such as a gas or liquid, which constitutes the mobile phase. Each component of the sample will interact differently with the stationary phase in the column and so move through the column at different speeds. As a result each will emerge from the column at different “retention times”. These separated species can then be analyzed absent any interference with other species in the sample. When the sample being introduced is a gas, the technique is called gas chromatography (GC), when the sample is in a liquid form the technique is called high performance liquid chromatography (HPLC). Either of these techniques is suitable for separating out the samples in the current device. An exemplary GC based explosive material detector is described by R. Batlle, et al., Anal. Chem. 75, 3137 (2003), the disclosure of which is incorporated herein by reference. In addition, to these standard chromatographic methods fast GC techniques could also be used to improve the speed of the analysis.

Another method of separating ionic species can be achieved through capillary electrophoresis. In this technique separation is achieved by the mobility differences imposed by the application of a potential difference to a drive fluid. A number of suitable capillary electrophoresis systems are disclosed by: B. R. McCord, et al., anal. Chim. Acta 288, 43 (1994); J. Wang, et al., Analyst (Cambridge, U.K.) 127, 719 (2002); and W. Thormann, et al., Electrophoresis 22, 4216 (2001), the disclosures of which are incorporated herein by reference.

Pyrolyzed Detection System

As shown in the schematic provided in FIG. 1, once the components of the sample have been separated in the separation step/system the components can be pyrolyzed into molecular fragments, and those fragments interrogated. The pyrolyzed detector comprises two basic systems, a pyrolyzer and a detector.

Turning first to the pyrolyzer, although any suitable pyrolyzer may be used, it is of critical importance that the pyrolyzer be efficient in fragmenting the separated components of sample. For example, in the simplest embodiment, the pyrolyzer could comprise a simple heated Nichrome wire. In such an embodiment, the temperature of the pyrolyzer would depend on the current delivered to the Nichrome wire. During operation, the current delivered to the pyrolyzer, and thus its temperature, would be set such that no absorbance due to the analyte remains, and only absorbance of fragments are observed. Suitable temperatures are easily obtainable from known reference materials. For example, the pyrolysis temperatures of most nitroarenes can be found in H. H. Hill, et al., Pure Appl. Chem. 74, 2281-2291 (2002), the disclosure of which is incorporated herein by reference.

Although such a simple pyrolyzer can be used, other more sophisticated pyrolyzers may also be used. For example, in one preferred embodiment of the current invention a catalytic pyrolyzer may be utilized. A catalytic pyrolyzer operates at much lower temperatures (275° C.), and produces fragments only from molecules that catalytically react with the pyrolysis device. For example, a catalytic pyrolyzer would produce NO only from targeted nitroorganic compounds. One exemplary catalytic pyrolyzer is described by D. H. Fine, et al., SPIE Subst. Detect. Syst. 2092, 131-136 (1993), the disclosure of which is incorporated herein by reference.

The second part of the pyrolyzing detector system is the detector. Regardless of the ultimate design of the detector, the pyrolyzed fragments of the components of the sample are passed into an analyzing cell where they can be interrogated by the detector. For example, in one preferred embodiment, the fragments are passed into a quartz cell, which can be interrogated via a spectroscopic detector such as an ultraviolet source. Although UV absorption spectra are typically broad and featureless, the small molecule fragments of such explosives formed in the pyrolyzer in accordance with the current invention are typically very sharp and well-defined. Exemplary GC-UV spectroscopic systems are described in further detail by: W. Kaye, Anal. Chem. 34, 287-293 (1962); T. Cedron-Fernandez, et al., Talanta 57, 555-563 (2000); H. V. Lagesson, et al., J. Chromatogr. 867, 187-206 (2000); M. J. McQuaid, et al., Appl. Spectrosc. 45, 916-917 (1991); A. D. Usachev, et al., Appl. Spectrosc. 55, 125-129 (2001); and W. A. Schroeder, et al., Anal. Chem. 23, 1740-1747 (1951), the disclosures of which are incorporated herein by reference.

It should be understood that although a UV source does have a number of advantages, including speed, simplicity, and accuracy, it is possible that other detection techniques, both spectroscopic and non-spectroscopic could be coupled with the separation system and pyrolyzer of the current invention, including, for example, infrared (IR), Fourier transform infrared (FTIR), and mass spectrometry (MS).

It should also be understood that any of the above techniques could be coupled with improved sample cells, enhanced sources, or advanced signal processing to increase the sensitivity of the device. For example, increases in sensitivity could be achieved by using a multi-pass cell, or a more sensitive detector.

(Optional) Orthogonal Detection System

As shown in FIG. 1, although the inventive explosives detector must include at least the pyrolyzing detector, the device may also include another orthogonal detector that would directly analyze the separated components from the separation system prior to pyrolysis. This separate analysis provides yet another dimension that can be combined with the retention time and data from the pyrolyzing detector of the multidimensional detector of the current invention to provide an even more sensitive fingerprint of each of the species found in the sample. It should be understood that any suitable method of analyzing the components produced by the separation system may be used. For example, any techniques, either spectroscopic or non-spectroscopic typically coupled with chromatographic techniques may be used including, IR, FTIR, GS, electron capture (ECD), chemiluminescence or thermal energy analysis (TEA), flame ionization (FI), thermal conductivity (TC), and surface acoustic wave (SAW). Exemplary GC-coupled devices were described by M. E. Walsh, Talanta 54, 427 (2001); E. J. Staples, et al., Pittsburgh Conference on Analytical Chemistry and Applied Spectroscopy, New Orleans, La., Paper No. 1583CP (1998); and D. P. Rounbehler, et al., First International Symposium on Explosive Detection Technology, Atlantic City, N.J. 703-713 (1991), the disclosure of which are incorporated herein by reference.

Analyzer

As shown in the schematic diagram an analyzer is provided to link all of the data from the various sources of the multidimensional detector of the current invention. Although any suitable multichannel analyzer may be used with the multidimensional detector of the current invention, as shown in FIG. 1, at the minimum it must be capable of monitoring and corresponding the retention times of the separated components of the sample and the data from the component fragments formed in the pyrolysis detector. In addition, as shown in some embodiments of the invention an optional orthogonal detector may be coupled with the separation system that would provide data on the unfragmented separated components provided by the separation system. In such an embodiment, these results would optimally be monitored and corresponded to the retention times provided by the separation system and the data of the fragments provided by the pyrolysis detector to provide another dimension for the multidimensional analysis of explosives detector system.

In its simplest form, the analyzer could be designed only to provide an indication when an explosive is present, without providing information about the identity or concentration of the explosive. In such an embodiment, the only relevant information would be to identify a fragment indicative of an explosive material, such as, for example, NO from an organonitrile explosive, at an appropriate retention time indicative of a known explosive material.

Although such a system would be inherently simple, the analyzer of the explosive detection system of the current invention could also be designed to provide both qualitative and quantitative information about the subject explosive. In such an embodiment, it would be necessary to have pre-stored calibration information in the analyzer. Any suitable calibration method and system could be used with the analyzer of the current invention. For example, in one embodiment a standard calibration protocol could be used such as those used for environmental detection or bulk detection, such as, for example EPA Method 8330 for environmental detection and the Assessment of Technologies Deployed to Improve Aviation Security: First Report (1999) for bulk assessments. Several studies model calibration protocols have been proposed for trace explosive detection including, G. A. Eiceman, et al., National Institute of Justice Report 100-99, NCJ 178261 (1999); and P. Kolla, Anal. Chem. 67(5) 184A (1995), the disclosures of which are incorporated herein by reference. Any of these proposed or model calibration methods could be used to provide suitable comparators for the qualitative and quantitative analysis of the multidimensional data produced by the detector of the current invention.

Alternatively, one could calibrate the current detector in lieu of certified standards by first exposing the detector to known concentrations of known explosives, such as through well characterized solid particles. Production of known explosives standards has been accomplished in various manners, including continuous thermal sources, transient methods using GC columns and injectors, and pulsed methods usually using a preconcentrator or precise mass of explosive material in a known volume. Some suitable methods are described by G. A. Eiceman, et al., Talanta 45, 57 (1997); M. G. Hartell, et al., Fifth International Symposium on Analysis and Detection of Explosives, Washington D.C., Paper No. 48 (1995); M. G. Harell, et al., Fifth International Symposium on Analysis and Detection of Explosives, Washington D.C., Paper No. 46 (1995); W. R. Stott, et al. Conference on Cargo Inspection Technologies, San Diego, Calif., SPIE Proc. 2267, 87 (1994); D. P. Lucero, et al., Advances in Analysis and Detection of Explosives, edited by J. Yinon (Kluwer Academic, Dordrecht) 485-502 (1993); J. P. Davies, et al., Advances in Analysis and Detection of Explosives, edited by J. Yinon (Kluwer Academic, Dordrecht) 513-532 (1993); J. P. Davies, et al., Anal. Chem. 65, 3004 (1993); E. E. A. Bromberg, et al., Proc. Int. Symp. Anal. Detect. Explos., 4th, London (Fluwer, Dordrecht) 473-484 (1992); B. T. Kenna, et al., Proc. Int. Symp. Explosive Detection Technology, 1st, Atlantic City (FAA) 510-517 (1992); S. J. Macdonald, et al., Proc. Int. Symp. Explosive Detection Technology, 1st, Atlantic City (FAA) 584-588 (1992); G. A. Reiner, et al., J. Ener. Mater. 9, 173-190 (1991); J. P. Davies, et al., Proc. SPIE 2092, 137 (1994); L. Elias, J. Test. Eval. 22, 280 (1994); and P. Neudorfl, et al., Proc. Int. Symp. Anal. Detect. Explos., 4th, (Kluwer, Dordrecht, London) 373-384 (1992), the disclosures of which are incorporated herein by reference.

In addition, to the above calibration methodologies, the analyzer may also be equipped with any suitable signal processing algorithms or techniques for further improving the resolution of the detection system. Exemplary signal processing techniques suitable for use with the current invention including, signal arithmetic like background subtraction or signal averaging, signal smoothing via signal to noise manipulation or optimization such as a rectangular or triangular smoothing function, differentiation such as derivative spectroscopy and trace analysis, resolution enhancement, and peak integration. Although a list of proposed techniques is provided it should understood that this is just a sampling of possible signal processing techniques that can be used with the current invention.

Incorporation into Devices

Although the above discussion has focused only on single units of the inventive detector, it should be understood that such a detector or an array of a plurality of such detectors could be incorporated into larger multi-purpose devices. For example, by combining multiple sensors with pattern recognition, a multi-component detection and analysis system could be constructed. Alternatively, a sensor or a plurality of such sensors could be manufactured into a microelectronic system to form a “sensor on a chip.” In either case any conventional supporting electronics, software and mechanical systems may be combined with one or more of the inventive multidimensional detectors of the current invention to form an integrated device.

### Exemplary Embodiment

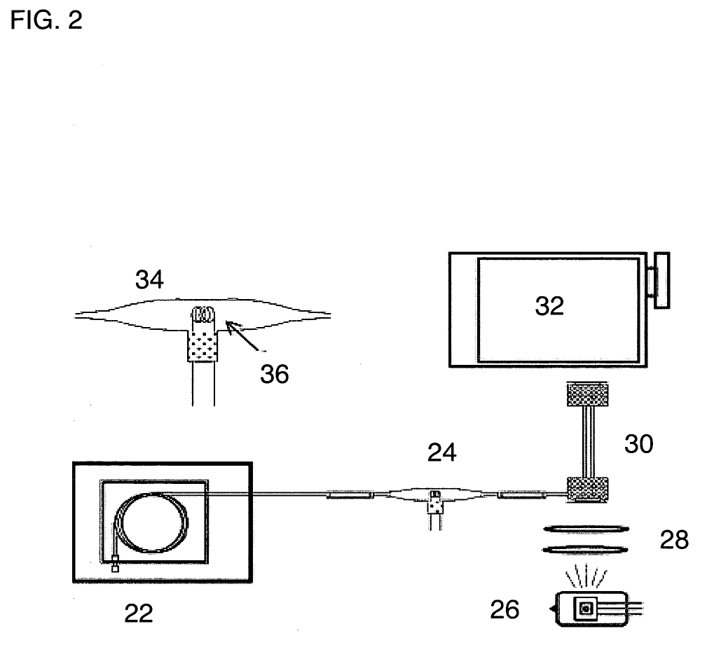

Although the components discussed above can be combined in any number of ways, in one preferred embodiment, the multidimensional detection system of the current invention employs an approach that combines gas chromatography as the separator with a pyrolyzed ultraviolet detector as the final detector. A schematic of such a device is shown schematically in FIG. 2. As shown in the figure, first the components of a sample having an explosive mixture are separated using a gas chromatograph instrument. Effluent from the gas chromatograph is then pyrolyzed. The molecular fragments produced from the pyrolysis, such as, for example, nitric oxide from a nitroorganic compound, is then detected by ultraviolet absorption spectroscopy. These small molecular fragments, such as nitric oxide exhibit more sharply banded characteristic spectrum than do the large complex starting products, enabling detection of very small concentrations.

GC-PUD of Nitroorganics

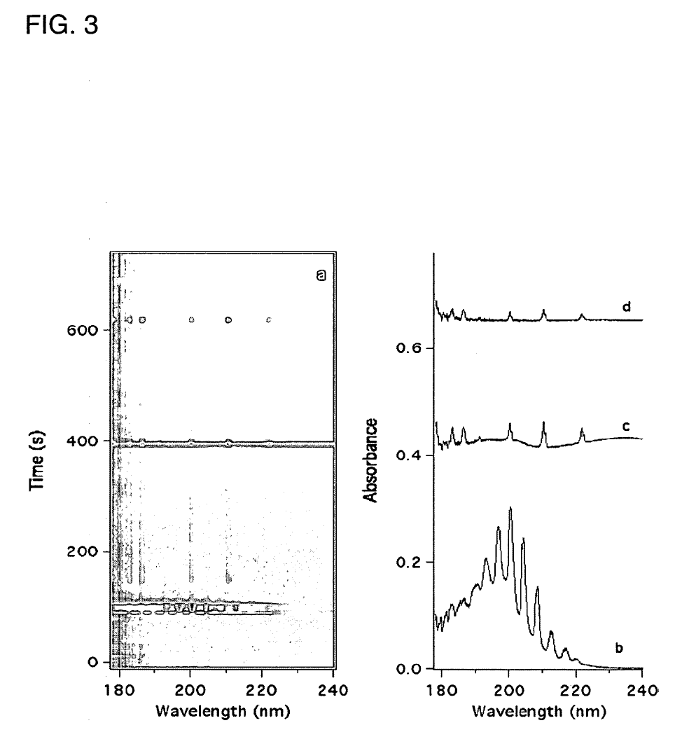

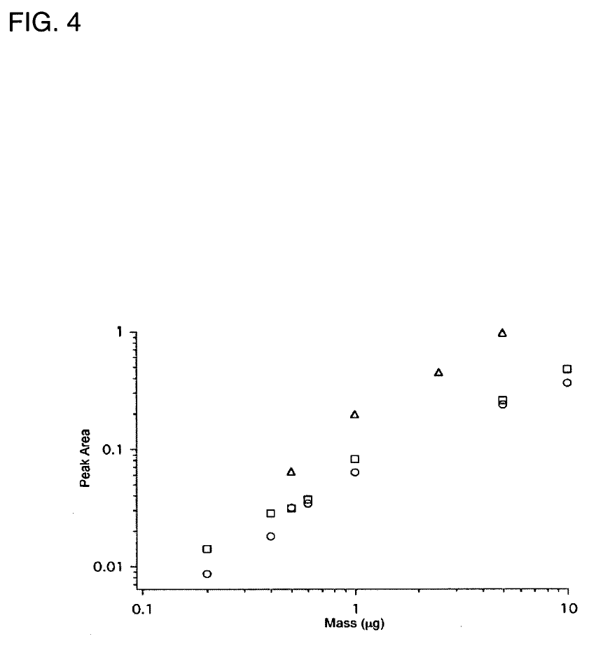

A detection system incorporating GC-PUD, was tested using the explosive materials nitrobenzene (1) and 2,4-dinitrotoluene (2), and with the nitramine explosive tetryl (3), the molecular formulas of which are shown below. As shown in the data provided in FIGS. 3 and 4, all three test explosives yield detectable NO on pyrolysis. Linearity of response and sensitivity are good, with a limit of detection of ˜50 ng for tetryl. Detection limits are 25 ng for nitrobenzene and 50 ng for 2,4-dinitrotoluene. Tetryl is detected with a detection limit of 50 ng.

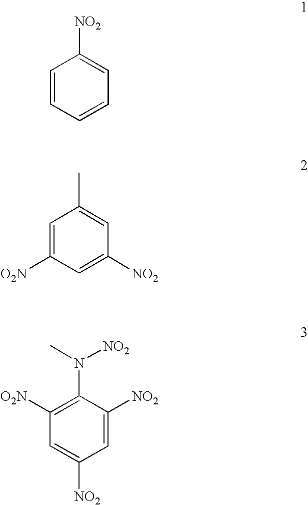

The apparatus used for the test experiments conforms to the schematic diagram provided in FIG. 2. A gas chromatograph (SRI Model 8610C) (22) was connected to a pyrolysis cell (24) via a heated stainless steel transfer line, usually held at 250° C. The pyrolysis cell was comprised of a Kimax glass envelope, ˜5 mm in diameter, inside of which was a coil of Nichrome wire (36). The tube was sealed using a high temperature ceramic putty. A current of 2-2.5 A was passed through the coil, heating it to a temperature of 900-1200° C. The temperature was measured with a Micro-Optical Pyrometer, manufactured by Pyrometer Instrument Co., Inc., Bergenfield, N.J. The gaseous products (34) from the pyrolysis flow were directed to a heated absorption cell. The cell itself consisted of two aluminum blocks supporting a quartz tube (3 mm OD) between them, with silica windows on either side. The tube served as both a light pipe and a conduit for the pyrolysis products. The cell had a pathlength of ˜6 cm, and was typically heated to 150° C. Residence time in the cell was approximately 3 s, so peak broadening due to the cell was eliminated.

The light from a 30 W deuterium lamp (Oriel 63163) (26) was coupled into the cell (30) using silica lenses (28). Unfocused light exiting the cell was directed into a Chromex 250 is imaging spectrograph (32) equipped with an Apex SPH-5 CCD detector. The resolution of the system was approximately 0.5 nm. The entire optical path, including the spectrometer, was purged with nitrogen to allow operation below 200 nm. Spectra from 180-240 nm ware acquired approximately every 1.5 seconds, with an integration time of 1 s.

The gas chromatograph uses a 100% methyl polysiloxane column (MXT-1 15 m×0.53 mm×5 μm film) with on-column injection. For nitrobenzene and 2,4-dinitrobenzene, the temperature of the GC oven was ramped from 50° C. to 250° C. at 15° C./min. The temperature program for tetryl was as follows: 100° C. for 2 minutes, then ramped at 10° C./min to 200° C., then ramped at 20° C./min to 250° C., and held for 5 minutes. Helium was used as the carrier gas with a source pressure of 5 psig. Nitrobenzene and 2, 4-dinitrobenzene (Aldrich) were used without further purification. Acetonitrile was obtained from EM Science. Tetryl was acquired as a 1 mg/mL solution in acetonitrile from Supelco.

FIG. 3a shows a representative 2-D chromatogram of 500 ng each of nitrobenzene (NB) and 2,4. dinitrotoluene (2,4-DNT) obtained by GC-PUD. The sample was injected as 1 μL of a 1 mg/M1 solution of NB and 2,4-DNT in acetonitrile. Clear signals are visible due to acetonitrile, NB, and 2,4-DNT at retention times of 100 s, 395 s, and 620 s, respectively. FIGS. 3b, c, and d show horizontal slices through the chromatogram at these retention times. The spectrum shown in FIG. 3b is identical to that of ammonia, indicating that ammonia is a product of the pyrolysis of acetonitrile. The spectra in FIGS. 3c and 3d match the spectrum of NO, indicating that NO is produced by the pyrolysis of NB and 2,4-DNT.

FIG. 4 shows the relationship between peak area and the mass of analyte injected into the gas chromatograph for NB, 2,4-DNT and tetryl. Peak areas were determined by taking a vertical slice through the 3-D chromatogram at the maximum of the 215 nm band of NO. This generates a chromatogram equivalent to an experiment where one monitors the absorbance of the eluent at 215 nm only. The 215 nm band was chosen because it has the largest absorbance in our experiment. The area of the peak representing the eluted compound was then determined for several injections of different masses of each compound.

The peak areas for all three analytes tested are linear with mass below approximately 5 micrograms. At higher concentrations, NB and 2,4-DNT showed a small negative deviation from linearity. The slope of the curves for NB and 2,4-DNT are essentially identical, while the slope for tetryl is significantly greater. The correspondence between the slopes of NB and 2,4-DNT shows that the number of nitro groups on the molecule is independent of the amount of NO generated by pyrolysis. The steeper slope of the curve for tetryl may be due to the presence of both nitro and nitramine functionalities in this compound, altering its pyrolysis behavior. Limits of detection (LOD), determined as three times the noise, were 50 ng for tetryl and 2,4-DNT, and 25 ng for nitrobenzene.

The test results prove that an inexpensive and simple multidimensional detection system can be implemented for the selective qualitative and quantitative detection of explosives in the presence of other organics by combining gas chromatography with pyrolysis ultraviolet detection. The GC-PUD system of the current invention is technically simple and provides a clear signal of the presence and concentration of explosive materials. Although the exemplary system was only designed to monitor NO fragments from the sample, other diagnostic pyrolytic reactions may also be probed with this technique. For instance, the production of ammonia from acetonitrile on pyrolysis suggests that all nitriles may form ammonia when pyrolyzed. The study of the pyrolysis products from a wide variety of compounds would enable the GC-PUD technique to be used for functional group analysis of complex mixtures.

The preceding description has been presented with reference to presently preferred embodiments of the invention. Workers skilled in the art and technology to which this invention pertains will appreciate that alterations and changes in the described structure may be practiced without meaningfully departing from the principal, spirit and scope of this invention.

Accordingly, the foregoing description should not be read as pertaining only to the precise structures described and illustrated in the accompanying drawings, but rather should be read consistent with and as support to the following claims which are to have their fullest and fair scope.

What is claimed is:

1. A multidimensional explosives detector comprising:
a separator having a fluid passage with an inlet and an outlet, said inlet being in fluid communication with a sample having at least two distinct components, said separator being designed to pass each component of the sample through the fluid passage at a rate dependent on the physical properties of the component such that each of the components from the sample pass through the outlet of the separator at a different time;
a pyrolysis detector in fluid communication with said outlet, the pyrolysis detector consisting of:
a pyrolyzer including a heated element capable of decomposing each component into a plurality of molecular fragments, and
a detector in fluid communication with said pyrolyzer such that each molecular fragment is analyzed by said detector; and
an analyzer in signal communication with at least the separator and the pyrolysis detector such that the time data from the separator and the fragment data from the pyrolysis detector are analyzed to provide a multidimensional data set indicative of the presence of an explosive material in the sample.

2. The multidimensional explosives detector of claim 1, wherein the separator is a gas chromatograph.

3. The multidimensional explosives detector of claim 1, wherein the pyrolyzer is a Nichrome wire.

4. The multidimensional explosives detector of claim 1, wherein the pyrolyzer is a catalytic pyrolyzer.

5. The multidimensional explosives detector of claim 1, wherein the detector is an ultraviolet detector.

6. The multidimensional explosives detector of claim 1, further comprising a secondary detector in fluid communication with the outlet of the separator, such that each separated component of the sample is analyzed prior to pyrolysis.

7. The multidimensional explosives detector of claim 6, wherein the secondary detector is selected from the group consisting of infrared (IR), Fourier transform infrared (FTIR), mass spectroscopy (MS), electron capture (ECD), chemiluminescence or thermal energy analysis (TEA), flame ionization (FI), thermal conductivity (TC), and surface acoustic wave (SAW).

8. The multidimensional explosives detector of claim 7, wherein the secondary detector is in signal communication with the analyzer to provide data on each separated component of the sample to the multidimensional data set.

9. The multidimensional explosives detector of claim 1, further comprising a sample preconcentrator in fluid communication with the inlet of the separator, such that the sample is concentrated prior to being introduced into the separator.

10. The multidimensional explosives detector of claim 9, wherein the preconcentrator comprises an enclosed volume having a sample absorbent material in contact with a flash heating system such that the sample is first absorbed onto the absorbent material and then flash heated within the enclosed volume to create a concentrated volume of sample.

11. The multidimensional explosives detector of claim 9, wherein the preconcentrator comprises a particle collector.

12. The multidimensional explosives detector of claim 1, wherein the analyzer further comprises a stored calibration standard for one of either the qualitative or quantitative analysis of the sample.

13. The multidimensional explosives detector of claim 1, wherein the analyzer further comprises at least one signal processing algorithm for processing the multidimensional data set.

14. A method for detecting explosives comprising:
separating a sample into its molecular components;
identifying a separation time for the molecular components;
pyrolyzing each of the components to obtain molecular fragments thereof;
identifying said molecular fragments; and
analyzing the data from the separation time identification and the molecular fragment identification for species indicative of an explosive material.

15. The method of claim 14, further comprising concentrating the sample prior to separating the sample.

16. The method of claim 14, further comprising identifying the separated components prior to pyrolysis.

17. The method of claim 14, further comprising comparing the analyzed data with a calibration standard to obtain at least one of either quantitative or qualitative information about the explosive material.

18. The method of claim 14, wherein the separating step is conducted using a gas chromatograph.

19. The method of claim 14, wherein the pyrolysis step is conducted using a catalytic pyrolyzer.

20. The method of claim 14, wherein the identification of the molecular fragments is conducted using an ultraviolet spectrometer.

21. The method of claim 14, wherein the identification of the components is conducted using a detector selected from the group consisting of infrared (IR), Fourier transform infrared (FTIR), mass spectroscopy (MS), electron capture (ECD), chemiluminescence or thermal energy analysis (TEA), flame ionization (FI), thermal conductivity (TC), and surface acoustic wave (SAW).

22. The method of claim 14, wherein the concentrating step is conducted using an enclosed volume having a sample absorbent material in contact with a flash heating system such that the sample is first absorbed onto the absorbent material and then flash heated within the enclosed volume to create a concentrated volume of sample.

23. The method of claim 14, wherein the concentrating step is conducted using a particle collector.
